(19) United States
(12) Patent Application Publication
(10) Pub. No.: US 2008/0097603 A1
(43) Pub. Date: Apr. 24, 2008

(54) Otologic Prostheses With Compressive Ossicular Engagement By An Elastic Structure And Method Of Implanting The Same

(76) Inventors: Robert Brosnahan, Germantown, TN (US); Richard W. Mendius, Collierville, TN (US)

Correspondence Address: GORDON & JACOBSON, P.C., 60 LONG RIDGE ROAD, SUITE 407, STAMFORD, CT, 06902, US

(21) Appl. No.: 11/749,936

(22) Filed: May 17, 2007

Related U.S. Application Data
(63) Continuation-in-part of application No. 11/551,839, filed on Oct. 23, 2006.

(51) Int. Cl. A61F 2/02

(52) U.S. Cl. 623/10

(57) ABSTRACT

An ossicular prosthesis has a shaft and an elastic engagement structure coupled to the shaft. The engagement structure at least partially defines an opening and is deformable to widen the opening to permit a portion of an ossicle to be received therein. In one embodiment, the engagement structure is formed to provide substantially three point contact against ossicular structure of various diameters. In accord with preferred materials, when the engagement structure is deformed to receive the portion of the ossicle, the stress in the engagement structure remains substantially constant throughout a majority of the deformation. The load required to deform the engagement structure into an open position is relatively small, facilitating the procedure as well as reducing the potential for damage to the intact ossicle.

### CROSS-REFERENCE TO RELATED APPLICATION

This application is a continuation-in-part of U.S. Ser. No. 11/551,839, filed Oct. 23, 2006, which is hereby incorporated by reference herein in its entirety.

### BACKGROUND OF THE INVENTION

1. Field of the Invention

This invention relates broadly to prostheses. More particularly, this invention relates to otologic prostheses for replacement of the ossicles of the middle ear.

2. State of the Art

Hearing is facilitated by the tympanic membrane transforming sound in the form of acoustic sound waves within the outer ear into mechanical vibrations within the ossicular chain of bones in the middle ear. These vibrations are transmitted to the footplate of the stapes where micro (vibration) or macro motion of this structure results in compression waves within the fluid of the inner ear, These compression waves lead to vibrations of the cilia (hair cells) located within the cochlear where they are translated into nerve impulses. These nerve impulses are sent to the brain via the cochlear nerve and are interpreted in the brain as sound.

Hearing efficiency can be lost to erosion of the ossicular bones: malleus, incus, and stapes. These bones can be completely replaced by a prosthesis (total ossicular replacement prosthesis, or TORP), or various combinations of these bones can be replaced for example only the malleus and incus can be replaced (by a partial ossicular replacement prosthesis, or PORP, that assumes the presence of an intact stapes), or just the stapes can be replaced. Several types of stapedial replacements have been designed, including bucket-handle and piston designs. In a piston prosthesis, a crook-like bight is provided for placement over the incus or malleus and a shaft extends from the bight to or in some cases through the footplate.

Piston prostheses are most commonly manufactured from metals or an assembly of a metal and a polymer. The metallic portion of a traditional piston is usually manufactured from stainless steel or titanium. By way of example, a conventional piston that is adapted to be attached to the incus generally includes a bight opening which is substantially larger than the diameter of the incus. The prosthesis is attached to the incus by positioning the bight over the long process of the incus and then plastically deforming the bight with an instrument to capture the incus and hold it in sufficiently close conformity to the incus for stability and vibrational transfer between the incus and the oval window. However, it may be difficult to crimp the bight in the small confines of the surgical area. Also, it is necessary to ensure that the bight is crimped evenly, but not too tightly, about the incus. Otherwise, pressure necrosis can occur to the ossicle.

U.S. Pat. Nos. 5,935,167 and 6,830,587 to a Wengen et al. describe piston prostheses made from titanium that do not require crimping. In each, a discrete clip constructed of round wire is connected with the upper end of the shaft of the prosthesis. The clip includes an integrated hinge-like clamp that extends outward from the clip defining two arced regions and two breaks about the circumference of the clamp: at the hinge-like clamp and an opposite opening for receiving the ossicle. With sufficient force, the clamp can be elastically deformed to force a portion of an ossicle, e.g., the long process of the incus or the malleus handle, between upper and lower portions of the clip where the ossicle is retained. However, in view of the combination of material and shape, pushing the clip over the ossicle, with the ossicle moving through the opening of the clip, requires a significant force to spread the clip apart. It is possible that this force could damage the ossicle. In addition, the arced regions have a radius designed to accept an incus of a single diameter. Other diameter incuses will not properly seat within the clip causing the incus to be subject to traumatic point contact.

The SMart™ stapes piston prosthesis from Gyrus ENT of Bartlett, Tenn., and generally described in U.S. Pat. Nos. 6,197,060 and 6,554,861 to Knox, provides a different approach that uses a shape memory alloy, for example Nitinol. This piston has the appearance of a conventional piston with its bight formed in a closed configuration. The bight is deformed into an opened configuration prior to implantation. The open bight is placed over the long process of the incus and localized heat is then applied to the bight with an instrument to cause the bight to reshape into a closed configuration in accord with the shape memory material's ability to recover its as-formed original shape through heating at a phase transformation temperature. The force applied by the bight to the ossicle can be of such magnitude so as to cause long term discomfort to the patient and/or pressure induced necrosis of the bone. Furthermore, tissue structures in the vicinity of the prosthesis can be damaged as a result of the excessive application of heat to activate re-shaping of the bight. Moreover, it is possible that the heat-activated closing of the bight will result in an incomplete coupling necessary for load transmission, still requiring traditional crimping methods to complete the procedure.

### SUMMARY OF THE INVENTION

It is therefore an object of the invention to provide an ossicular prosthesis that requires fewer steps to apply to the appropriate ossicle.

It is another object of the invention to provide an ossicular prosthesis that will always conform to an ossicle without necessitating any crimping.

It is a further object of the invention to provide an ossicular prosthesis that distributes to more than two locations the load transfer about the ossicle to which it is coupled.

It is also an object of the invention to provide an ossicular prosthesis that requires minimal load to apply the prosthesis to an ossicle.

In accord with these objects, which will be discussed in detail below, an ossicular prosthesis is provided. In accord with one embodiment of the invention, the prosthesis has a shaft with a lower portion for placement at the oval window or stapes footplate and an upper portion coupled to a curved bight adapted to compressively engage an ossicular portion such as the long process of the incus or the malleus handle. The embodiments of the invention preferably include a bight (hook or shepherd's crook) made from a superelastic metal alloy or other suitable metal having requisite elasticity that provides a controlled compressive force against an ossicular structure.

In one embodiment, the bight is preferably curved through at least 180° for engaging about the portion of the ossicle and at least partially defines an opening. The bight is elastically deformable to widen the opening to permit the portion of the ossicle to be received therein. When made from a superelastic alloy, the bight is deformed to receive the portion of the ossicle, and after an initial linear loading, the stress in the bight remains substantially constant throughout its deformation as the bight is loaded with non-linear behavior in accord with a property of the superelastic metal alloy. The circular configuration of the bight loads the ossicle relatively evenly about its circumference with a large contact area between the bight and the ossicle, rather than at diametric or potentially traumatic point contact as is done in some prior art devices. Furthermore, the bight has a low profile that hugs the ossicle and reduces interferences and potential protrusion through the tympanic membrane.

In accord with another aspect of this embodiment, a handle is provided on the bight at a location displaced from the shaft and directed outward from the bight for handling the prosthesis with instrumentation.

In another embodiment, the bight is an extension of a piston shaft thus forming a one-piece bight and piston shaft. The piston shaft is preferably round in cross-section and transitions into a preferably flattened ribbon for the bight. The bight is preferably superelastic in manufacture. The bight is formed to contact an ossicular structure at three substantially evenly spaced locations. To that end, the bight includes three sides with two curved portions therebetween. The curved portions having a radius of curvature, and the opening through the narrowest portion of the bight is preferably greater than two times the radius. The bight extends into a laterally extending support on which the prosthesis can be temporarily supported on the ossicular structure during implantation. The configuration of the bight evenly loads the ossicle at three spaced apart locations and is adaptable to maintain such contact at various ossicle diameters.

In other embodiments, prostheses are provided with elastic elements that act in a compressive manner against various specific ossicular structure.

In view of the superelastic property of the preferred material for the bight (or other compressive ossicular engagement structure), in each embodiment the load required to deform the bight into an open position is relatively small. This reduces potential damage to the intact ossicle as well as facilitates the procedure. In addition, the bight always springs back over the ossicle, without requiring any secondary crimping.

Additional objects and advantages of the invention will become apparent to those skilled in the art upon reference to the detailed description taken in conjunction with the provided figures.

### BRIEF DESCRIPTION OF THE DRAWINGS

FIG. 1 is a side elevation of a stapedial piston prosthesis according to a first embodiment of the invention.

FIG. 2 is a graph showing the superelastic stress-strain curve of superelastic nickel titanium alloy.

FIG. 3 is a side elevation of a stapedial piston prosthesis according to a second embodiment of the invention.

FIG. 4 is a side elevation of a stapedial piston prosthesis according to a third embodiment of the invention.

FIG. 5 is a view of the stapedial piston prosthesis of the third embodiment of the invention, rotated 90° relative to FIG. 4.

FIG. 6 is a perspective view of a tympanoplasty partial ossicular replacement prosthesis (PORP).

FIG. 7 is a perspective view of an incudo-stapedial prosthesis according to the invention.

FIG. 8 is a side elevation of a stapedial piston prosthesis according to another embodiment of the invention.

FIG. 9 is a view of the stapedial piston prosthesis of FIG. 8, rotated 90° relative to FIG. 8.

### DETAILED DESCRIPTION OF THE PREFERRED EMBODIMENTS

Turning now to FIG. 1, an ossicular prosthesis 10 according to the invention is shown. The embodiment of the prosthesis is a stapedial piston for placement at or between the oval window or stapes footplate and the long crus (or process) of the incus. The piston 10 includes a curved bight (also referred to as a crook or hook) 12 and a shaft 14. The bight 12 extends into a substantially straight upper portion 16 of the stem 14. The upper portion 16 is coupled to (or expands into) a larger diameter preferably cylindrical lower portion 18 of the shaft 14 for placement on or through the stapes footplate.

The bight 12 preferably extends through of a curve α greater than 180°, preferably at least 240°, and more preferably at least 270°, and is open at 20 to include access for receiving a portion of the ossicle about which the bight is to be engaged. The diameter of the bight is preferably approximately the same dimension or slightly smaller than the intended ossicular portion to enable slight compression about the ossicle. The bight 12 includes a free end 22 that is angled to guide the portion of the ossicle therein as the bight is maneuvered toward the ossicle. It is appreciated that the bight could be positioned and/or configured such that the opening 20 is defined between either angled free end 22 of the bight and the upper end of the upper portion 16 of the shaft, or free end 22 and another portion of the bight distanced from the upper portion 16 of the shaft. While the bight 12 is shown as crook-shaped, it is appreciated that it may be provided with another shape capable of securely engaging the target ossicular portion with a compressive force.

The bight 12 and upper portion 16 of the shaft 14 are preferably a unitary construct made of the same material, and most preferably a nickel-titanium alloy (Nitinol) wire. The wire is preferably approximately 0.10 mm in diameter. Alternatively, the bight 12 may be made from a Nitinol wire and the upper portion 16 of the shaft 14 may be made from non-superelastic material, including a metal, metal alloy or polymer and coupled to the bight, and the two may be coupled together, e.g., with an adhesive, metallurgical, or mechanical coupling. It is also possible that bight 12 and the upper portion 16 maybe manufactured from Nitinol wire and is coupled to a metallic or polymeric lower portion 18 via and adhesive, metallurgical or a mechanical coupling.

In accord with a preferred aspect of the invention, the Nitinol wire of the bight 12 and upper portion 16 of the shaft 14 have superelastic behavior. Referring to FIG. 2, as the prosthesis 10 is applied to an ossicle and the bight 12 is forced open, thereby increasing the strain in the Nitinol wire, after an initial short period of linear stress, the stress in the bight 12 remains substantially constant across a loading plateau. Such behavior is termed non-linear superelasticity. This superelastic behavior is observed at a temperature above the austenite finish temperature of Nitinol, and arises from the stress-induced martensitic transformation on loading and spontaneous reversion of the transformation upon unloading. Thus, as also shown in FIG. 2, upon unloading, the stress also remains substantially constant across an unloading plateau. This behavior is distinct from the shape memory behavior of Nitinol which is an ability to recover an original shape upon heating through a phase transformation temperature. The production of Nitinol with superelastic or shape memory behavior is known in the art.

A handle 24 is optionally coupled to the bight 12 and extends radially outward from a center of the bight. The handle 24 is displaced from the shaft 12 for handling the prosthesis with instrumentation. In addition, the handle 24 is also preferably displaced on an opposite side of the opening 20 relative to the axis A, to allow the handle 24 to operate as a lever to bend the bight into an open configuration, if desired.

The lower portion 18 of the shaft may be made from metal, such as titanium, titanium alloy, or stainless steel, or a polymer such as a polytetrafluoroethylene, and is larger in diameter than the wire shaft 16. The larger diameter is preferred, if not necessary, for implanting at the target anatomy and permitting the prosthesis to cause the required movement at the oval window to restore hearing. The overall length of the shaft 12 is generally between 3.5-7 mm so that the shaft fits the anatomy between the incus or the malleus and the oval window.

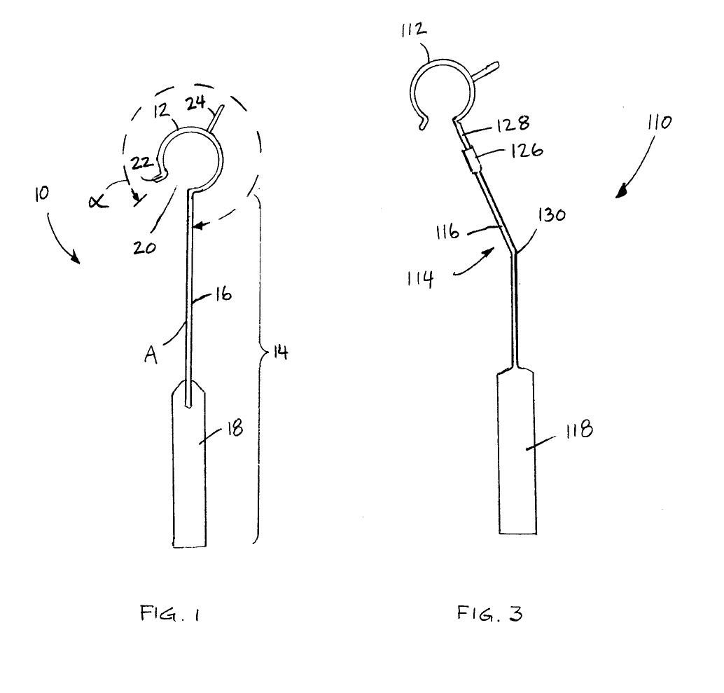

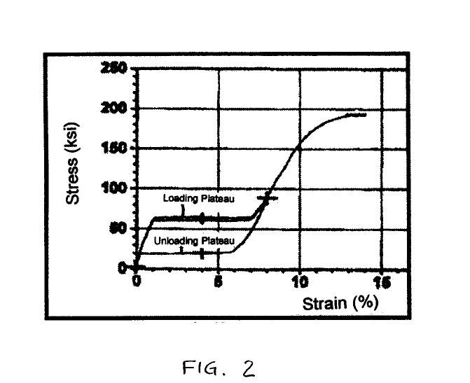

Turning now to FIG. 3, a piston prosthesis 110 having a bight 112 similar to bight 12 is shown. The shaft 114 of the piston 110 is preferably slightly longer (e.g., total length up to 5-7 mm). In addition, substantially the entirety of the shaft 114 is made from a non-superelastic material, such as titanium, stainless steel, or non-superelastic nickel-titanium alloy. Further, the piston shaft and bight may be made from a unitary element that is processed such that the bight portion has superelasticity and the shaft portion is plastically deformable. This allows the shaft 114 to be plastically deformable to define an angled bend, e.g., at location 130, by the surgeon or the manufacturer such that the prosthesis is extendable between the oval window and the malleus handle. The shaft 114 is preferably constructed of a common material and unitary construct from larger diameter lower portion 118 to the stepped down upper portion 116. The upper portion 116 defines an enlarged upper mount 126 for receiving an extension 128 of the bight 112. The extension 128 of the bight 112 is preferably heat melted into the mount 126, although other coupling means can be used. Alternatively, the lower portion 118 may be separately formed from upper portion 116, with the lower portion formed from the same or a different metal, metal alloy or a polymer from the upper portion.

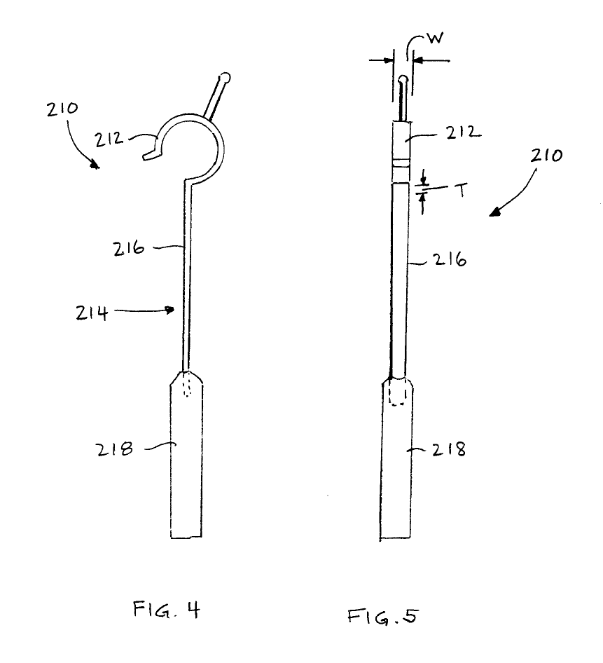

Turning now to FIGS. 4 and 5, a stapedial piston 210 substantially similar to piston 10 is shown, with the two pistons being distinguished from each other in the structure of the bights 212, 12. Piston 210 has a bight 212 that is comprised of a superelastic Nitinol ribbon having a width W substantially greater than its thickness T, whereas bight 12 of piston 10 is a shaped wire of relatively uniform diameter. The ribbon extends from the bight 212 into the upper portion 216 of the shaft 214. The ribbon is coupled to a lower portion 218 of the shaft that may be manufactured from a non-superelastic material. The ribbon provides enhanced stability and high contact area against the ossicular structure (to reduce stress), whereas the shaft is round so as to provide uniform stiffness in all planes.

Referring now to FIG. 6, a partial prosthesis 310 for tympanoplasty is shown. Prosthesis 310 includes an oval or circular flat disc-like head 312 with spokes 312a for mating with the tympanic membrane, a clip-like shoe 316 for placement over the stapes, and a shaft 314 or other space extending means for spanning between the head and the shoe. The shaft 314 typically has a length between 1.75-3.5 mm (and preferably not exceeding 5 mm). The clip 316 is defined by at least two prongs, and preferably more than four prongs 318 adapted to stably engage the stapes under compression. The clip 316 defines an opening 320 through which the stapes can be received under compression. The prongs 318 are preferably made from a superelastic metal alloy such as Nitinol. While the head 312 and shaft 314 also may be manufactured from a superelastic alloy, it is possible for them to be made from non-superelastic materials, such as titanium, titanium alloy or stainless steel, and polymers.

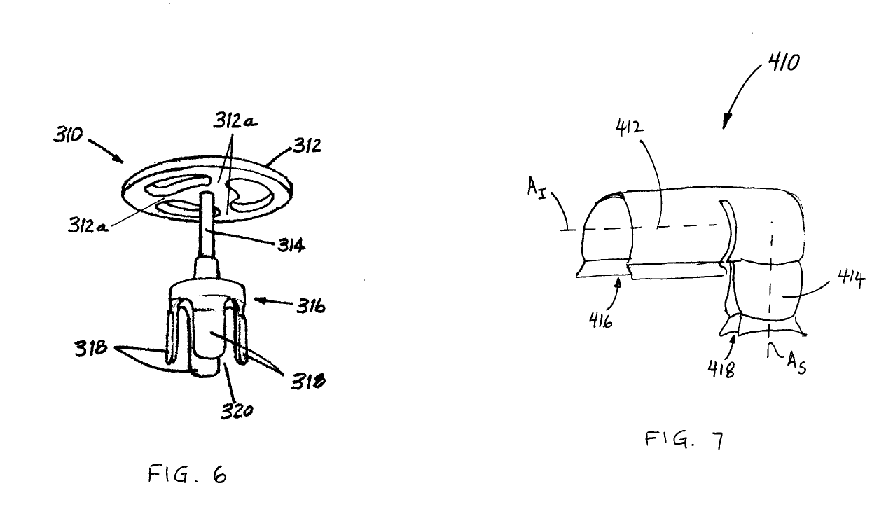

Turning now to FIG. 7, another embodiment of a device according to the invention is shown. The device 410 is an incudo-stapedial joint (ISJ) with two separate engagement structures 412, 414 preferably made from superelastic nickel titanium alloy. Each engagement structure is elastically deformed when placed on an incus and a stapes respectively, and then engages a separate portion of the respective ossicles under compression. The engagement structure are provided on different axes A_{I} (axis of incus) and A_{S} (axis of stapes) which are oriented transversely to each other. However, both engagement structures have openings 416, 418 at a lower end.

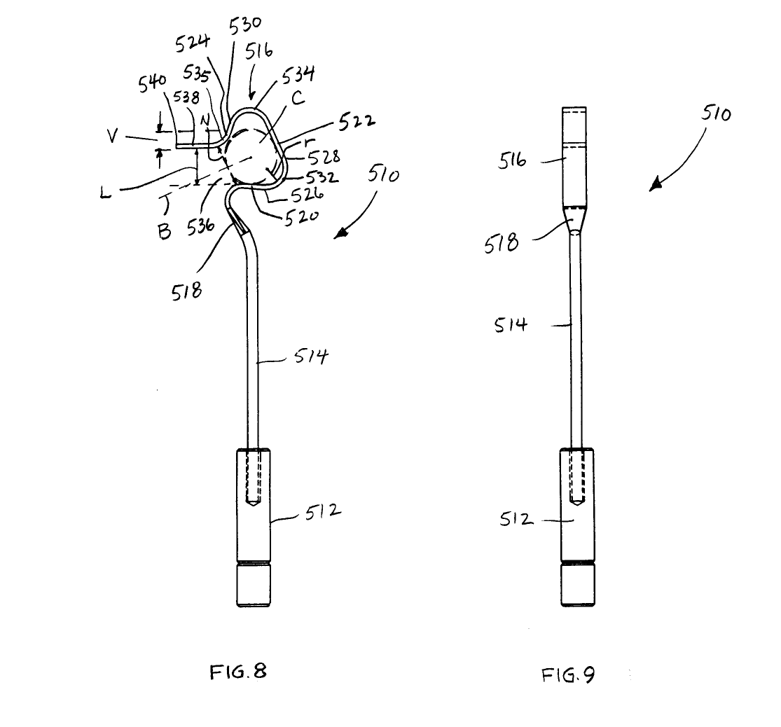

Referring now to FIGS. 8 and 9, another ossicular prosthesis 510 according to the invention is shown. The prosthesis 510 is a stapedial piston prosthesis for placement between the oval window (or stapes footplate) and the incus. The prosthesis includes a body 512, a piston shaft 514 and a bight 516. The body 512 is preferably cylindrical in shape and may be made from any suitable material, including metals, e.g., titanium, or plastics, e.g., Teflon. The piston shaft 514 is preferably coupled to the body 512 by crimping or bonding. The piston shaft 514 extends into the bight 516, with the two structures formed from a unitary piece of material. The material is preferably superelastic Nitinol. The piston shaft 514 is a wire generally circular or square in cross-section. The wire extends into the bight 516 at the upper end of transition 518 at which the shaft material is flattened into a larger width ribbon that is formed into the bight shape. The ribbon construct at the bight 516 provides increased stability, with an increased surface area contacting the incus, whereas the rounder diameter of the piston shaft 514 prevents inadvertent deformation to the piston shaft and facilitates coupling the piston shaft to the body 512.

The bight 516 is formed to contact an ossicular structure at three preferably substantially evenly spaced and preferably blunt “point” locations (i.e., 120°±15° apart) 520, 522, 524 upon final implantation. To that end, the bight includes three sides 526, 528, 530 with two curved portions 532, 534 therebetween, and another convexly curved portion 535 (relative to the interior of the bight) toward the end of the bight. Curved portions 532, 534 have a radius of curvature and, and for each curved portion 532, 534, the opening 536 into the bight 516 at narrow N is preferably greater than two times the respective radius. The radius for each curved portion 523, 534 is preferably the same, r=0.008±0.001 inch. Point 520 is along side 526, point 522 is along side 528, and point 524 is along curved portion 535.

An elastic bight formed to have three “point” locations of contact can accommodate incuses having various diameters and engage each equally well. The “point” locations are along the substantially flat sides or the radiused convex curves, and due to the superelastic nature of Nitinol, are atraumatic to the incus. The term “point” does not necessarily mean a sharp feature about the bight, but rather a small area of contact. As discussed in more detail below, when manufactured from a superelastic alloy, the configuration of the bight 516 evenly loads an ossicle preferably only at three “point” locations and is further adapted to maintain such contact at various ossicular diameters. The three “points” of contact 520, 522, 524 ensure that the prosthesis 510 is not too loose on the ossicle, which would otherwise reduce vibrational energy and may cause pain, or too tight, which can lead to bone necrosis. Furthermore, in view of the stress-strain curve of superelastic Nitinol, discussed in more detail, a very low load is required to elastically deform the bight to deform about the diameter of any incus.

The bight 516 extends through the convexly curved portion 535 into a laterally extending support 538 terminating in a free end 540. During implantation the prosthesis can be temporarily supported at support 538 on ossicular structure while the lower end of the body 512 is seated at the footplate or oval window. The support 538 is vertically offset from the top of the largest circle C that can be inscribed within the non-deformed bight 516 by a dimension V, which is preferably less 0.75 mm, and more preferably less than approximately 0.2 mm. If the offset V is too large, once the bight is placed over the incus, the implant may seat too deeply on the oval window and result in permanent vertigo.

The opening into the bight 516 is preferably substantially lateral, extending at a direction at or between a parallel to support 538 and bisector B. In addition, the vertical dimension L at the opening is 85±10 percent of the dimension of the opening parallel to bisector B; i.e., the dimension across narrows N. The above construction minimizes vertical displacement V as the bight is pushed laterally over the incus for engagement.

A similar design to bight 516 can be used with a longer optionally bent piston shaft (as described with respect to prosthesis 110) for application as mallear piston prosthesis. The same advantages result. Moreover, the shaft associated with this style of piston is generally slightly longer than an incus piston (functional length of incus piston shaft 4.5 mm-5.5 mm vs. functional length of the mallear piston shaft 5.5 mm-6.5 mm). Moreover, intraoperatively, the shaft of a malleus piston is typically bent in 2 planes to fit the anatomy. The mallear piston may include a superelastic segment extending from approximately 1 mm of the shaft immediately below the bight and through the bight, and a second segment extending to a body. The second segment preferably has been subject to less cold work than the superelastic segment such that the two segments each have a different modulus of elasticity. The elasticity modulus of the second segment allows the second segment to be plastically deformable intraoperatively to permit the surgeon to shape the prosthesis to fit the anatomy of the patient.

In each embodiment, the superelastic behavior of the bight, clip, or other engagement structure permits such engagement structure to be deformed with low load to permit entrance of an ossicular portion, of various diameters, within the engagement structure. The engagement structure is deformable to widen the opening to permit a portion of an ossicle (e.g., long process of incus, malleus head, capitulum of the stapes) to be received therein and, in accord with the superelastic behavior of the Nitinol material, after a short period of linear stress, the stress in the engagement structure remains substantially constant during such deformation. Such period of linear stress is substantially short in duration such that the engagement structure is subject to substantially constant stress (in a non-linear relationship to the applied strain) throughout the majority of the engagement structure deformation. Then, as the ossicular portion is received in the engagement structure, the engagement structure does not forcibly snap on the ossicle, but rather will result in a low but constant compressive force thereabout it. In addition, the superelastic behavior also substantially evenly distributes force about the ossicle, rather than concentrate the loading force. Further, the engagement structure will always attempt to recover to assume its original shape which conforms to the ossicle for enhanced stability and security and load transfer. In addition, the prosthesis maintains a low profile, reducing the potential for interference within other ossicles, or the potential for protrusion through the tympanic membrane.

There have been described and illustrated herein several embodiments of a ossicular prosthesis, and particularly a prostheses for stapedioplasty and tympanoplasty and a method of implanting the same. While particular embodiments of the invention have been described, it is not intended that the invention be limited thereto, as it is intended that the invention be as broad in scope as the art will allow and that the specification be read likewise. Moreover, while specific embodiments of prostheses have been disclosed, it is appreciated that other embodiments for prostheses for replacements of one or more ossicles or the joints therebetween can be provided, with such embodiments having an engagement structure that partially surrounds, particularly with three-point contact, and engages a portion of an ossicle under compression. Furthermore, while a superelastic alloy is preferred, another biocompatible material with high elasticity can also be used. For example, grades 3 or 4 commercially pure (CP) titanium has sufficient stiffness and hardness to function as a highly elastic spring such that it can be readily deformed and then spring back to engage an ossicle without high load to the ossicle or plastic deformation. While such grades of titanium do not have the same elasticity and substantially constant stress during loading as does superelastic nickel-titanium, both are resilient to mishandling and retain their formed shape. Also, structural elements and materials of the various embodiments can be combined and/or used interchangeably. It will therefore be appreciated by those skilled in the art that yet other modifications could be made to the provided invention without deviating from its spirit and scope as claimed.

1. An ossicular prosthesis for positioning between a portion of an ossicle and either a stapes footplate or an oval window, comprising:
an elongate body portion having first and second ends;
a smaller diameter shaft extending upward from second end of said body;
a bight extending from said upper portion of said shaft, said bight shaped to contact an ossicular structure at three locations post-implantation, and said bight including non-ossicular contacting portions between each of said three locations,
wherein the bight at least partially defines an opening, the bight being deformable to widen the opening to permit the portion of the ossicle to be received therein, and when the bight is deformed to receive the portion of the ossicle, the stress in the bight remains substantially constant throughout a majority of its deformation.

2.-4. (canceled)

5. An ossicular prosthesis according to claim 1, wherein:
said shaft and said bight are each portions of a common element, and said shaft and said bight each have a different modulus of elasticity.

6. An ossicular prosthesis according to claim 1, wherein:
said body is cylindrical.

7. (canceled)

8. An ossicular prosthesis according to claim 1, wherein:
said locations are substantially evenly spaced apart.

9. An ossicular prosthesis according to claim 1, wherein: said locations are blunt.

10. An ossicular prosthesis according to claim 1, wherein:
said bight includes three substantially straight sides, and a curved portion located at the junction of adjacent sides.

11. An ossicular prosthesis according to claim 10, wherein:
said bight includes an opening at a narrow and each of said curved portions has a respective radius, and for each curved portion a dimension across said narrow is greater twice said respective radius.

12. An ossicular prosthesis according to claim 1, further comprising:
a laterally extending support extending from said bight and terminating in a free end.

13. An ossicular prosthesis according to claim 8, wherein:
said three locations define a circle of maximum diameter that can be inscribed within said bight, said bight extends into a laterally extending support terminating in a free end, and said support is vertically offset from the top of said circle by an amount that is less than 0.75 mm.

14. An ossicular prosthesis according to claim 1, wherein:
said bight is made from grades 3 or 4 CP titanium.

15. An ossicular prosthesis for positioning between a portion of an ossicle and either a stapes footplate or an oval window, comprising:
an elongate body portion having first and second ends;
a smaller diameter shaft extending upward from second end of said body;
an elastic metal alloy bight extending from said upper portion of said shaft for engagement about a portion of the ossicle, said bight including two straight sides, a curved portion therebetween, and an opening including a narrows, said bight being deformable to widen the opening at the narrows to permit the portion of the ossicle to be received therein.

16. An ossicular prosthesis according to claim 15, wherein:
said bight includes three straight sides and two curved portions, one each at the junction of adjacent sides, said curved portions each curved about a respective radius, for each curved portion said narrows having a dimension at least twice the respective radius.

17. An ossicular prosthesis according to claim 15, wherein:
when the bight is deformed to receive the portion of the ossicle, the stress in the bight remains substantially constant throughout a majority of its deformation.

18. An ossicular prosthesis according to claim 15, wherein:
said shaft comprises a wire and said bight comprises a ribbon.

19. An ossicular prosthesis according to claim 15, wherein:
said shaft and bight are each portions of a common element.

20. An ossicular prosthesis according to claim 19, further comprising:
a laterally extending support extending from said bight and terminating in a free end.

21. An ossicular prosthesis according to claim 15, wherein:
said bight defines a circle of maximum diameter that can be inscribed within said bight, said bight extends into a laterally extending support terminating in a free end, and said support is vertically offset from the top of said circle by an amount that is less than 0.75 mm.

22. An ossicular prosthesis according to claim 15, wherein:
said bight is made from superelastic Nitinol or grades 3 or 4 CP titanium.

23. An ossicular prosthesis for positioning between a portion of an ossicle and either a stapes footplate or an oval window, comprising:
an elongate body portion having first and second ends;
a smaller diameter shaft extending upward from second end of said body;
an elastic metal alloy bight extending from said upper portion of said shaft for engagement about a portion of the ossicle, said bight defining an opening including a narrows, said bight being elastically deformable to widen the opening at the narrows to permit the portion of the ossicle to be received therein, and said bight structured to contact the portion of the ossicle at only three spaced apart locations.

24. An ossicular prosthesis according to claim 23, wherein:
when the bight is deformed to receive the portion of the ossicle, the stress in the bight remains substantially constant throughout a majority of its deformation.

25. An ossicular prosthesis according to claim 23, further comprising:
a laterally extending support extending from said bight and terminating in a free end.

26. A method of implanting an ossicular prosthesis, comprising:
a) providing an ossicular prosthesis having an engagement structure defining an opening through which a portion of an ossicle can be received therein, the engagement structure adapted to apply a compressive force at only three spaced apart locations to restrain the ossicle received therein, the engagement structure made from an elastic metal or metal alloy; and
b) implanting the ossicular prosthesis, wherein during the implantation the engagement structure is deformed to widen the opening to permit the portion of the ossicle to be received therein, and when the engagement portion is deformed to receive the portion of the ossicle, the engagement portion contacts the ossicle at the three spaced apart locations.

27. A method according to claim 26, wherein:
the stress in the engagement structure remains substantially constant throughout a majority of its deformation, and after the portion of the ossicle is received therein the engagement portion applies a compressive force only to the three spaced apart locations on the portion of the ossicle.
